(19) United States
(12) Patent Application Publication
(10) Pub. No.: US 2011/0270305 A1
(43) Pub. Date: Nov. 3, 2011

(54) SURGICAL THREAD, IN PARTICULAR FOR PREVENTING PUNCTURE CHANNEL BLEEDING, AND A METHOD FOR PRODUCING SAME

(75) Inventors: Sven Oberhoffner, Weinstadt (DE); Heinrich Planck, Nurtingen (DE); Erhard Müller, Stuttgart (DE)

(73) Assignee: ITV DENKENDORF PRODUKTSERVICE GMBH, Denkendorf (DE)

(21) Appl. No.: 13/128,168

(22) PCT Filed: Nov. 6, 2009
(86) PCT No.: PCT/EP09/07947

§ 371 Date: Jul. 21, 2011

(30) Foreign Application Priority Data
Nov. 6, 2008 (DE) 10 2008 057 214.4

(51) Int. Cl. A61B 17/04; B29C 47/06

(52) U.S. Cl. 606/228; 264/172.15

(57) ABSTRACT

A surgical thread avoids puncture channel bleeding, and has a polymeric core and a polymeric sheath surrounding the polymeric core, wherein the polymeric sheath is swellable in bodily fluids.

### RELATED APPLICATIONS

This is a §371 of International Application No. PCT/EP2009/007947, with an international filing date of Nov. 6, 2009 (WO 2010/052004 A2, published May 14, 2010), which is based on German Patent Application No. 10 2008 057 214.4, filed Nov. 6, 2008, the subject matter of which is incorporated by reference.

### TECHNICAL FIELD

This disclosure relates to a surgical thread which has a polymeric core-sheath construction and which is particularly useful for preventing puncture channel bleeding, a corresponding surgical kit, production methods for the surgical thread and the use of the thread as surgical suture.

### BACKGROUND

Wounds are typically closed in state of the art surgical care using thread-shaped sutures combined with surgical needles. As the needle passes into a biological tissue it creates a so-called “puncture channel” wherethrough the suture is subsequently pulled. Since in most cases the needle diameter far exceeds that of the suture, the puncture channel created by the needle is not completely filled up by the suture. This can lead to so-called “puncture channel bleeding,” and this can lead to complications in the case of cardiovascular stitches in particular. In addition, a puncture channel which is not completely filled by the suture offers colonization opportunities to infectious pathogens, which greatly heightens the risk of post-operative infection.

US 2007/0275034 A1 discloses a suture based on an absorbable, amphiphilic block copolymer having a central block of polyethylene glycol and terminal blocks of glycolide, lactide, ε-caprolactone, p-dioxanone, trimethylene carbonate and/or morpholinedione. One disadvantage with this is the actually poor swellability of the suture in bodily fluids, which greatly limits its possible use for the purpose of preventing puncture channel bleeding. Nor in fact is the possible occurrence of puncture channel bleeding addressed at all in the US application.

WO 2006/138300 A2 discloses inter alia a bioswellable suture having a layered construction. The swellable properties of the suture are due to a hydrophilic outer layer. This hydrophilic outer layer is produced by graft polymerization and possesses a polymeric network with ionic groups. Owing to the osmotic pressure of the surrounding bodily fluid, the network which contains ionic groups and constitutes essentially a superabsorbent attached to the thread core by a covalent bond ensures very rapid swelling of the suture in the body. A possible consequence of this is that, as the suture is being pulled through a tissue, it already assumes a significantly larger diameter than a needle secured to the end of the suture. This necessitates a high pull-through force which can lead to increased traumatization of the tissue. Nor is knot repositionability likely to be achievable. Furthermore, the ionic groups of the covalently attached superabsorbent are in direct contact with the bodily fluids, blood in particular, and may affect blood coagulation adversely, or cause undesired ion exchange processes, for example, as a result of interactions, and thus impair important processes taking place in the body. Moreover, graft polymerization is a generally costly and inconvenient method of production. A further disadvantage is that graft polymerization is generally not capable of providing a unitary thickness of layer, and this can lead to nonuniform tendencies for the suture to expand in physiological media. This in turn restricts possible uses for the purposes of preventing puncture channel bleeding.

It could therefore be helpful to provide a surgical thread which prevents puncture channel bleeding without having known disadvantages. At the same time, it could also be helpful that the thread should ensure enhanced knot security and have an improved knot hold. It could further be helpful to provide production methods for the surgical thread which are very simple and inexpensive to carry out compared to conventional methods.

### SUMMARY

We provide a surgical thread that avoids puncture channel bleeding including a polymeric core and a polymeric sheath surrounding the polymeric core, wherein the polymeric sheath is swellable in bodily fluids.

We also provide a surgical kit including the surgical thread and at least one surgical needle.

We further provide a method for producing the surgical thread, wherein a polymeric thread core component and a polymeric sheath component swellable in bodily fluids are coextruded to form threads having a polymeric core and a polymeric sheath swellable in bodily fluids which surrounds the core.

We still further provide a method for producing the surgical thread, wherein a thread-shaped polymeric thread core component is coated by sheath extrusion with a polymeric sheath component swellable in bodily fluids to form threads having a polymeric core and a polymeric sheath swellable in bodily fluids which surrounds the core.

We further yet provide a method for producing the surgical thread, wherein a thread-shaped, polymeric thread core component is dipped into an aqueous solution of a crosslinkable, water-soluble and polymeric sheath component, pulled through an aqueous solution of a crosslinkable, water-soluble and polymeric sheath component or sprayed with a solution of a crosslinkable, water-soluble and polymeric sheath component, and the sheath component is crosslinked to form threads having a polymeric core and a polymeric sheath swellable in bodily fluids which surrounds the core.

### DETAILED DESCRIPTION

The thread comprises a surgical thread, preferably a surgical suture, particularly for preventing puncture channel bleeding, having a polymeric core and a polymeric sheath surrounding the polymeric core (core-sheath construction), wherein the polymeric sheath is swellable, or made to be swellable, in bodily fluids.

In other words, we provide a surgical thread, preferably in the form of a surgical suture, having a polymeric core-sheath construction, the sheath of which is swellable, or made to be swellable, in bodily fluids. The swellable embodification of the sheath causes the surgical thread to expand, preferably in a controlled manner, on contact with bodily fluids, particularly blood, and thereby for the thread to acquire an altogether increased diameter. When the thread is accordingly pulled through a puncture channel formed by a surgical needle and knotted, the puncture channel bleeding which generally occurs in the process causes the sheath to swell. The resulting expansion or diameter enlargement of the thread leads with particular advantage to a complete and impervious closure of the puncture channel. This makes it impossible for infectious pathogens to invade the puncture channel, distinctly reducing the risk of post-operative infection. In fact, it is perfectly possible for the swellability of the sheath to cause the thread to expand more than corresponds to the diameter of the puncture channel. As a result, the polymeric sheath presses up against the puncture channel wall to provide particularly good sealing of the puncture channel.

A further advantage of the thread is that blood which has penetrated into the swellable sheath generally coagulates, which keeps the puncture channel dry and, for example, prevents the formation and possibly accumulation of exudate in the puncture channel. A further advantage resulting from the special swelling properties of the sheath is that the swelling does not take place too quickly, i.e., essentially not before placing of the knot. In addition to improving knot security and knot hold, the repositionability of the knot is also not adversely affected as a result.

A bodily fluid is in principle any fluid occurring in the human and/or animal body and also any fluid which is actually body-compatible. Accordingly, bodily fluids may be water, blood, lymph fluids, pus fluids, exudates, urine or physiological buffer solutions. Preferably, however, bodily fluids are human and/or animal blood.

We provide in principle for the polymeric core of the thread to be surrounded by the polymeric sheath to a partial extent only. In general, however, the polymeric core of the thread is surrounded by the polymeric sheath completely, i.e., over its entire surface.

Preferably, the polymeric core and the polymeric sheath touch along a common interface (area between polymeric core and polymeric sheath) without the polymeric core and the polymeric sheath being attached to each other by a covalent bond. In other words, it is particularly preferable for the attachment between the polymeric core and the polymeric sheath to be free of covalent bonds. Attachment between the polymeric core and the polymeric sheath can be based on purely forces of adhesion, for example. Particularly, the polymeric core and the polymeric sheath are adhered together along a common interface. It is particularly preferable for the surgical thread to be present as extrusion thread, particularly as coextrusion thread or sheath extrusion thread.

Further preferably, the thread is present as a bicomponent thread. A bicomponent thread is a thread having a polymeric core and a polymeric sheath surrounding the polymeric core, the sheath and the core generally each being formed of a different polymeric material.

Preferably, the polymeric sheath has an absorbency for bodily fluids which corresponds to 3 to 80 times and particularly 5 to 40 times its own dry weight.

Preferably, the polymeric sheath includes additives swellable in bodily fluids, preferably superabsorbents. The use of superabsorbents is a particularly advantageous way to bring about an additional improvement in the swellability of the polymeric sheath and hence also a greater expansion in the thread diameter. The superabsorbents can be between 1 and 100 μm and particularly 5 and 50 μm in particle size. Advantageously, the contemplated superabsorbents have biocompatible properties. Suitable superabsorbents can have an absorbency for fluids which corresponds to more than 100 times their own dry weight. Preferably, the polymeric sheath includes additives swellable in bodily fluids, particularly superabsorbents, in a proportion between 2% and 20% by weight and particularly 3% and 8% by weight, based on the overall weight of the polymeric sheath. Preferred superabsorbents are selected from the group consisting of polyacrylates, polymethacrylates, starch, hydroxyethylcellulose, hyaluronic acid, linear polysaccharides, gelatin, carrageenan, pectins and mixtures thereof.

In addition to the additives mentioned in the preceding section, the thread may include further additives, in particular active ingredients. These active ingredients may be selected from the group consisting of active antimicrobial, disinfecting, anti-inflammatory, growth-promoting and odor-controlling ingredients.

When the polymeric sheath does contain swellable additives, it may be advantageous for the polymeric sheath itself to have a certain elasticity. This is because it can thereby be prevented that the swelling additives cause the sheath to become brittle and acquire cracks or even spall off material. When swellable additives are present in the sheath, it may further be advantageous for the polymeric sheath to have a certain hydrophilicity, preferably without itself being water-soluble. As a result, water in bodily fluids can diffuse into the polymeric sheath and to the swellable additives and initiate the swelling process. The hydrophilicity of the sheath can thus be used to set the diffusion rate and the time course of swelling. It is therefore particularly preferable for the polymeric sheath to be made hydrophilic, water-insoluble and at least partly elastic and more particularly wholly elastic.

The polymeric sheath may include a hydrophilic, water-insoluble and preferably at least partly elastic and more particularly wholly elastic polymer. The polymer may in principle be a homo-, co-, tri-, tetrapolymer or the like. Copolymers are to be hereinafter understood as meaning polymers composed of two or more different monomeric units. More particularly, the polymer may be present as a block co- or terpolymer or as a segmented polymer. The polymer may further be an elastomer, more particularly a thermoplastic elastomer. The elastomer may be present in uncrosslinked form, particularly in nonvulcanized form. Preferably, the polymer is selected from the group consisting of polyurethanes, polyester-ethers, mixtures thereof and copolymers thereof, particularly from the group consisting of segmented polyurethanes, segmented polyester-ethers, mixtures (blends) thereof and copolymers thereof. Polyurethanes may particularly comprise linear and preferably aliphatic polyurethanes. An example of a suitable polyurethane is the polyurethane used by B. Braun Melsungen AG under the internal designation Vasomer®.

The polymeric sheath may particularly include a polymer matrix swellable in bodily fluids. The polymeric sheath may be formed of a polymer matrix swellable in bodily fluids. It is particularly preferable for the polymer matrix to include a hydrophilic, water-insoluble and at least partly elastic and more particularly wholly elastic polymer and additives swellable in bodily fluids, preferably superabsorbents. In general, the polymer matrix itself is formed by a hydrophilic, water-insoluble and at least partly elastic and more particularly wholly elastic polymer, in which case additives swellable in bodily fluids, preferably superabsorbents, are present embedded into the polymer matrix. In addition to the advantages described above, this example has the advantage that incorporation of the swellable additives, particularly superabsorbents, in a polymer matrix substantially avoids direct contact between ionic groups of the swellable additives, particularly superabsorbents, and bodily fluids, particularly blood. With regard to further features and details, the observations made hereinabove are referenced.

To increase the hydrophilicity of the polymeric sheath, it may further be provided for the polymeric sheath to include a polymer blend. The polymer blend preferably includes a hydrophilic, water-insoluble and preferably at least partly elastic and more particularly wholly elastic polymer and also a hydrophilic and preferably water-soluble polymer. The hydrophilic and preferably water-soluble polymer is preferably selected from the group consisting of polyethylene glycol, polypropylene oxide, polytetramethylene oxide, polyvinylpyrrolidone, polyvinyl alcohol, mixtures thereof and copolymers thereof. To further increase the hydrophilicity of the polymeric sheath, the polymers described hereinabove may also be grafted or copolymerized with monomers having ionic groups, for example, carboxylate and/or sulfonate groups.

The polymeric sheath may be present as a hydrogel or alternatively is convertible into a hydrogel on contact with bodily fluids. A hydrogel, unlike superabsorbents, generally has essentially no charge-bearing groups and is obtainable, for example, via physical or chemical crosslinking of water-soluble or at least highly hydrophilic polymers. Suitable methods of crosslinking will be described in greater detail in what follows.

It is therefore further preferable for the polymeric sheath to include a crosslinkable, particularly chemically and/or physically crosslinkable, polymer, in which case the crosslinkable polymer is generally a water-soluble or at least highly hydrophilic polymer. A water-soluble polymer is generally rendered water-insoluble by crosslinking Examples of suitable polymers can be selected from the group consisting of polyvinyl alcohol, polyvinylpyrrolidone, mixtures thereof and copolymers thereof. Mixtures may additionally also contain polyethylene glycol. We may further provide for the polymeric sheath to include an already crosslinked, particularly chemically and/or physically crosslinked, and preferably water-insoluble polymer.

Advantageously, the polymeric core of the thread may not be swellable in bodily fluids. Preferably, the polymeric core of the thread is formed of a polymer not swellable in bodily fluids. This ensures that the surgical thread as a whole retains some basic mechanical strength, particularly with regard to linear breaking strength, breaking extension, knot breaking strength and knot breaking extension. Preferably, the polymeric core of the thread is formed of a polymer from the group consisting of polyolefins, polyesters, polyamides, mixtures thereof and copolymers thereof. For example, the polymer for the polymeric core of the thread is selected from the group consisting of polyethylene, polypropylene, polyethylene terephthalate, mixtures thereof and copolymers thereof.

The polymeric sheath may have a fraction between 15% and 60% by volume and particularly 20% and 45% by volume, based on the overall volume of the thread. Accordingly, the polymeric core can have a fraction between 85% and 40% by volume and particularly 80% and 55% by volume, based on the overall volume of the thread.

The thread preferably has a circular cross section. Other cross-sectional shapes are also conceivable. For example, the thread can have an oval, triangular/trilobal, square, trapezoidal, rhomboid, pentagonal/five-cornered, hexagonal/six-cornered, star-shaped or cruciform cross section. Such cross-sectional shapes are readily realizable, for example, using appropriate extrusion rams, which are obtainable with any desired cross-sectional shape specific to the customer.

The polymeric sheath may have a unitary or uniform thickness of layer. More preferably, the polymeric sheath has a radius fraction between 8% and 37% and particularly 11% and 26%, based on the overall radius of a cross-sectionally circular thread. The polymeric core can have a radius fraction between 92% and 63% and particularly 89% and 74%, based on the overall radius of a cross-sectionally circular thread.

The polymeric core may further have a diameter between 0.07 and 1 mm and more particularly 0.1 and 0.7 mm. The thread itself preferably has a diameter between 0.1 and 1.5 mm and more particularly 0.15 and 1.0 mm.

The surgical thread may in principle be present as a monofilament or as a multifilament. Preferably, the surgical thread is present as a monofilament. The surgical thread may further be present as a so-called “pseudomonofilament.” A pseudomonofilament is a surgical thread whose polymeric core is formed either of two, three or more, more particularly a multiplicity, of generally very fine monofilaments, or of a multifilament yarn, which are or is, respectively, conjointly surrounded by the polymeric sheath.

The surgical thread preferably comprises surgical suture.

To additionally minimize the risk of puncture channel bleeding and post-operative infection, it may additionally be provided for the thread to have a tapered diameter in the region of one end or both ends. This makes it possible for the thread to be combined, to particular advantage, with a surgical needle actually designed for smaller thread diameters. In this way, the thread diameter can be brought into line with the needle diameter even in the dry state of the thread. A needle to thread diameter ratio of <2:1 may be provided, preferably 1:1. For example, the tapered diameter in the region of the thread ends can correspond to the diameter of the polymeric core of the thread, whereas the remaining thread regions preferably have the original thread diameter (including the thickness of the polymeric sheath). More particularly, the polymeric core of the thread can have a diameter which corresponds to the diameter of a needle hole and its overall diameter (including the thickness of the polymeric sheath) corresponds to the diameter of the needle. As a result, the puncture channel created by the needle can be completely and more particularly sealingly filled by the untapered regions of the thread, which is advantageous. A further advantage described in this section is that the thread diameter, owing to the swellable properties of the polymeric sheath, distinctly exceeds the needle diameter and hence the diameter of the puncture channel formed by the needle, on contact with bodily fluids. This in turn creates a particularly impervious closure of puncture channels. To taper the diameter, the thread can be exfoliated for example in the region of its ends.

The region of the thread ends may have the polymeric sheath completely exfoliated. To exfoliate the thread ends, thermal methods can be used, for example, laser methods. Such exfoliating techniques commend themselves particularly when the polymeric sheath melts at lower temperatures than the polymeric core. A further suitable exfoliating technique for a thread whose sheath includes a crosslinkable and water-soluble polymeric material is for the thread ends to be immersed in water or an aqueous solution, for example, over the length of a needle hole, and for the remaining regions of the thread then to be subjected to crosslinking. The transition from the original diameter of the thread to a tapered diameter in the region of the thread ends can be made abrupt or continuous, more particularly in the form of a gradient. To form a gradual transition, extrusion is a particularly suitable technique. For instance, the hauloff speed when extruding a thread can be varied, periodically in particular. This can be accomplished, for example, by modulating the circumferential speed of the godet responsible for hauling off the thread. Alternatively, additional godets can be inserted between the extrusion die and the hauloff godet.

At least one end and more particularly one end of the thread may be attached to a surgical needle. However, it is also possible for both the thread ends to be attached to a surgical needle. For attachment to a surgical needle, typically part of the thread is introduced into a hole drilled into the needle for this purpose and the needle is subsequently crimped/beaded in the region of the drilled hole.

It may further be provided for the thread to be present in sterilized and more particularly end-itemed form. To sterilize the thread, any method of sterilization known to a person skilled in the art in principle can be used, more particularly γ-sterilization, electron beam radiation, x-ray irradiation, ethylene oxide gasification and/or plasma sterilization. To end item the thread, it is generally trimmed to a particular length and subsequently packed sterile in suitable packaging, for example blister packaging.

A further aspect relates to a surgical kit comprising the thread and also at least one surgical instrument of insertion, preferably a surgical needle. It may perfectly well be provided for the kit to include two surgical needles each intended for securement to one end of the thread. With regard to further features and details concerning the kit, the description hereinabove is referenced in full.

We further provide a method for producing the surgical thread, wherein a polymeric thread core component and a polymeric sheath component swellable in bodily fluids are coextruded to form threads having a polymeric core and a polymeric sheath swellable in bodily fluids which surrounds the core.

In an alternative production method for the surgical thread, a thread-shaped polymeric thread core component is coated, preferably by sheath extrusion, with a polymeric sheath component swellable in bodily fluids to form threads having a polymeric core and a polymeric sheath swellable in bodily fluids which surrounds the core. Apart from a sheath extrusion, the thread-shaped, polymeric component of a thread can also be coated with the polymeric sheath component swellable in bodily fluids by dipping and/or spraying. For example, the thread core component can be dipped into an aqueous solution of the sheath component to become coated therewith. Alternatively or additionally, the thread core component can also be coated by pulling it through an aqueous solution of the sheath component. Alternatively or additionally, it may further be provided that the thread core component be sprayed with an aqueous solution of the sheath component.

For coextrusion, the thread core polymer and the sheath polymer are generally melted in extruders suitable for this, twin-screw extruders, for example, which may be co- or counter-rotating, and extruded under elevated temperature and pressure from a shape-conferring two-material die at a very uniform rate. Coextrusion can be carried out as a so-called “bicomponent extrusion.” In bicomponent extrusion, the melts for the thread core and the polymeric sheath each consist of a different polymer. Bicomponent extrusion is a particularly economical method of production. More particularly, bicomponent extrusion is capable of producing threads having particularly good core-sheath adherence. Sheath extrusion has the advantage that mono- or multifilaments, more particularly multifilament yarns, can also be used as thread core component. When multifilaments are chosen for the thread core component, pseudomonofilaments are obtainable in this way.

Preferably, a hydrophilic water-insoluble and preferably at least partly elastic and more particularly wholly elastic polymer is used for the sheath component. Optionally, polymer blends can also be used. With regard to further features and details, particularly concerning suitable polymers and/or polymer blends, reference is made in full to the description hereinabove.

Swellable additives, preferably superabsorbents, may be incorporated into a hydrophilic, water-insoluble and at least partly elastic and more particularly wholly elastic polymer to provide the sheath component. It is particularly preferable to incorporate the swellable additives in the polymer in the form of a masterbatch or compound. A masterbatch or compound is a concentrate of the swellable additives in a suitable polymer. The masterbatch or compound is obtainable on kneaders or twin-screw extruders, for example. When the swellable additives are superabsorbents, it can be advantageous to mechanically comminute these, for example, via ball mills or cutting mills, before use.

In a further alternative production method for the surgical thread, a thread-shaped, polymeric thread core component is dipped into an aqueous solution of a crosslinkable, water-soluble and polymeric sheath component, pulled through an aqueous solution of a crosslinkable, water-soluble and polymeric sheath component or sprayed with an aqueous solution of a crosslinkable, water-soluble and polymeric sheath component, and the sheath component is crosslinked to form threads having a polymeric core and a polymeric sheath swellable in bodily fluids which surrounds the core.

Again, this method of production makes it possible to use mono- or multifilaments and more particularly multifilament yarns for the thread core component. To improve the solubility of the sheath component, the aqueous solution may contain proportions of organic solvents. Examples of suitable solvents are methanol, ethanol, isopropanol, acetone and/or mixtures thereof. The proportion of the sheath component in the aqueous solution can be between 3% and 30% by weight and more particularly 7% and 25% by weight, based on the total weight of the aqueous solution. After the thread core component has been immersed in and/or pulled through the aqueous solution of the sheath component, more particularly after the crosslinking of the sheath component, the produced thread is generally dried. Drying can take place under heating, for example, in a thermal oven suitable for this purpose, or in vacuo, optionally likewise under heating.

Preferably, the sheath component is subjected to crosslinking, more particularly chemical and/or physical crosslinking Crosslinking, more particularly radiative crosslinking, is preferably effected at elevated temperatures, more particularly in a temperature range between 50° C. and 95° C., such that the mobility of the polymer chains of the sheath may be increased. Preferably, crosslinking is effected after the coextrusion or sheath extrusion or after the dipping into the aqueous solution or after the pulling through the aqueous solution of the sheath component.

Crosslinking may be effected using a crosslinking agent from the group consisting of carbodiimides, divinyl sulfone, N-methylenebisacrylamide, epichlorohydrin, bi- or oligofunctional aldehydes, polyfunctional polyaldehydes, bi- or oligofunctional carboxylic acids, bi- or oligofunctional esters, polyfunctional polycarboxylic acids, polyfunctional polyesters and borax. An example of a suitable bifunctional aldehyde is glutaraldehyde. A suitable bifunctional carboxylic acid, in particular dicarboxylic acid, is maleic acid (cis-butenedioic acid).

Crosslinking may be carried out free-radically, particularly under action of UV light and preferably in the presence of suitable free-radical initiators. For example, polyvinylpyrrolidone can be crosslinked by exposure to UV light having a wavelength of about 360 nm and in the presence of a suitable initiator, for example disodium 4,4′-diazidostilbene-2,2′-disulfonate. An alternative free-radical method of crosslinking envisages temperatures between 70 and 90° C. in the presence of small amounts of a peroxide, for example. t-butyl peroxypivalate or H₂O₂/CuCl₂. This method of crosslinking is particularly useful for high molecular weight polyvinylpyrrolidone grades.

Crosslinking may be effected by exposure to ionizing radiation, for example, (3-radiation, γ-radiation, electron beam radiation or x-ray radiation. To crosslink polyvinyl alcohol or polyvinylpyrrolidone, for example, a radiation dose between 10 and 50 kGy (kilogray) and preferably 20 and 40 kGy (kilogray) can be used. Ionizing crosslinking and more particularly radiation-induced crosslinking may very advantageously also effect sterilization of the thread at the same time.

With regard to the production method for the surgical thread wherein the thread core component is dipped into an aqueous solution of the sheath component and/or pulled through an aqueous solution of the sheath component, crosslinking can also be effected in the aqueous solution. The thread core component can be crosslinked during or shortly after the immersion and/or pull-through phase. A suitable method of crosslinking envisages a pH change, optionally with simultaneous temperature change, of the aqueous solution or an immersion into an alkaline solution of a thread core component sheathed with the sheath component. For example, crosslinking in an aqueous solution containing low molecular weight polyvinylpyrrolidone grades can be achieved by raising the pH to >11 and at elevated temperatures.

Crosslinking the sheath component, particularly by the crosslinking methods previously described, can be used to specifically influence/control the properties of the thread. A high degree of crosslinking minimizes the swellability and more particularly the absorbency of the polymeric sheath for bodily fluids. On the other hand, however, a higher degree of crosslinking of the sheath also contributes to a higher mechanical stability of the thread than a low degree of crosslinking. In addition, crosslinking the sheath component can lead to a uniform/homogeneous distribution of any additives, preferably superabsorbents, present in the polymeric sheath.

In general, the threads are drawn after coextrusion. In the case of sheath extrusion or of the production method wherein the thread core component is dipped into and/or pulled through an aqueous solution of the sheath component, by contrast, the thread core component will generally already be in the drawn form. Drawing can be done continuously or batchwise. In continuous drawing, the threads are generally led over a system of rollers or godets which may have different speeds of rotation. Usually, each subsequent roller or godet will have a higher speed of rotation than the preceding roller or godet of the drawing system. In batchwise drawing, by contrast, the threads are generally clamped between suitable holding or fixing elements, for example, jaws, of a tensioning device and subsequently drawn. Drawing can also be done under heating and/or in vacuo.

With regard to further features and details of the production methods, more particularly concerning polymers useful for the thread core and sheath components, the description hereinabove is referenced in full.

Finally, we provide for the use of the surgical thread as suture or to be more precise surgical suture, particularly for preventing puncture channel bleeding, preferably in cardiovascular surgery. With regard to further features and details concerning the surgical thread, reference is again made to the description hereinabove.

Further features will be apparent from the following description of preferred examples. Individual features herein may each be actualized on their own or combined with each or one another.

### Example 1

### Sheathing a Pet Monofilament with a Compound Consisting of 15% by Weight of Superabsorbent and 85% by Weight of an Aliphatic Polyurethane (Vasomer®)

T5066F superabsorbent from Stockhausen was sieved to remove everything but a fraction having particle sizes <50 μm for use. A compound for sheathing a monofilament of polyethylene terephthalate (PET) was produced using a twin-screw extruder having two dosing devices. The dosing stations here were adjusted such that 15% by weight of the superabsorbent and 85% by weight of the polyurethane were conveyed per unit of time. The zones of the extruder were heated to temperatures between 130 and 160° C. The spinhead temperature was likewise 160° C. At a screw speed of 40 rpm a virtually homogeneous compound was extruded through a 3.0 mm die and the strand was hauled off at 3 m/min. After cooling by leaving to stand at room temperature, the strands were pelletized. The average pellet diameter was 2.3 mm. The actual sheath extrusion of the PET monofilament with a diameter of 0.31 mm took place on a single-screw extruder with a 0.175 cubic centimeter spinpump equipped with a sheathing die. The spinhead temperature was set to 160° C. as in the case of the production of compound. The extrusion speed, i.e., the pull-through speed of the PET monofilament, was 30 m/min. The spinpump speed was 3.2 rpm. After cooling along a 15 m sector, the sheathed monofilament was wound up. The diameter of the sheathed monofilament was 0.35 mm, which corresponds to a suture of USP size 2-0. The almost white sheath of the monofilament exhibited low roughness.

### Example 2

### Sheathing a PET Multifilament Braid

A multifilament braid of polyethylene terephthalate (PET) having a USP diameter of 0.32 mm was sheathed under the same conditions as described in Example 1. The measured pseudomonofilament diameter was likewise 0.35 mm. The pseudomonofilament proved to be distinctly slacker in flexure than the sheath monofilament.

### Example 3

### Bicomponent Extrusion of Monofilament with Polypropylene Core and a Sheath of Compound Consisting of 15% by Weight of Superabsorbent and 85% by Weight of an Aliphatic Polyurethane (Vasomer®)

A bicomponent extrusion plant consisting of a single-screw extruder, a twin-screw extruder and in each case a spinpump (0.25 cubic centimeter) per stream and a bicomponent spinhead with core-sheath die (1.2 mm, L/D=8) was used to produce a monofilament having a core of polypropylene and a sheath of superabsorbents and polyurethane. The sheath was produced using the compound described in Example 1. For this, the compound ran on the twin-screw extruder. The polypropylene used was of the type Borealis HC 11 5 FB and had an MFI of 2.8 (230° C./2.16 kg).

TABLE 1
extrusion parameters

| | Single-screw | Twin-screw |
|---|---|---|
| | extruder | extruder |
| zone 1 temperature [° C.] | 200 | 130 |
| zone 2 temperature [° C.] | 220 | 150 |
| zone 3 temperature [° C.] | 230 | 150 |
| line temperature [° C.] | 230 | 160 |
| spinhead temperature [° C.] | 195 | |
| spinpump [rpm] | 21.8 | 5.5 |

| | |
|---|---|
| die-bath separation [cm] | 4 |
| quench bath temperature [° C.] | 20-22 |
| haul off [m/min] | 10.0 |
| outer diameter [mm] | 0.93 |
| core diameter [mm] | 0.82 |

Before determination of the diameter with a double axis laser measuring instrument, the monofilament was dried on a heated drum at 70° C. and in a vacuum of 0.5 mbar overnight. The core-sheath monofilament was then drawn in two stages, the second stage being used for relaxation to increase flexibility.

TABLE 2
drawing parameters

| | |
|---|---|
| feed godet 1 [m/min] | 2 |
| slot heater 1 [° C.] | 100 |
| godet 2 [m/min] | 16 |
| slot heater 2 [° C.] | 115 |
| godet 3 [m/min] | 14 |
| overall draw ratio | 7 |
| monofilament outer diameter [mm] | 0.35 |
| monofilament inner diameter [mm] | 0.31 |

### Example 4

### Extruding a Monofilament with a Core of Polypropylene and a Sheath Consisting of a PVA-PVP Blend (70% by Weight/30% by Weight)

The twin-screw extruder of the bicomponent monofil plant of Example 3 was equipped with two dosing stations to feed the extruder with polyvinyl alcohol (PVA) and polyvinylpyrrolidone (PVP) in a ratio of 70/30 (% by weight/% by weight). Mechanical mixing and direct feeding of the extruder was not possible on account of the different particle sizes (PVA as granules, PVP as fine powder). Otherwise the experimental setup corresponded to the setup described in Example 3. In Mowiflex TC232 PVA from Kuraray and Luvitec VA64 PVP from BASF, two modified types of polymer were chosen as being particularly good for thermoplastic processing.

TABLE 3
extrusion parameters

| | Single-screw | Twin-screw |
|---|---|---|
| | extruder | extruder |
| zone 1 temperature [° C.] | 200 | 180 |
| zone 2 temperature [° C.] | 220 | 180 |
| zone 3 temperature [° C.] | 230 | 180 |
| line temperature [° C.] | 230 | 180 |
| spinhead temperature [° C.] | 195 | |
| spinpump [rpm] | 21.8 | 5.5 |

| | |
|---|---|
| die-bath separation [cm] | 4 |
| quench bath temperature [° C.] | 20-22 |
| haul off [m/min] | 10.0 |
| outer diameter [mm] | 0.94 |
| core diameter [mm] | 0.82 |

Before determination of the diameter with a double axis laser measuring instrument, the monofilament was dried on a heated drum at 70° C. and in a vacuum of 0.5 mbar overnight. The core-sheath monofilament was then drawn in two stages, as described in Example 3, the second stage being used for relaxation to increase flexibility. The external diameter was 0.36 mm and the core diameter was 0.31 mm.

### Example 5

### Sheathing a PET Monofilament with a Blend Consisting of PVA and PVP (70% by Weight/30% by Weight)

The sheathing extruder described in Example 1 was equipped with two dosing stations like the bicomponent extruder described in Example 4 such that polyvinyl alcohol (PVA) and polyvinylpyrrolidone (PVP) may be fed to the extruder in the desired weight ratio. The extruder and spinhead temperatures were set to 180° C. and 185° C., respectively. A monofilament of polyethylene terephthalate (PET) having a diameter of 0.29 mm was used. The spinpump speed of the extruder was set to 4.8 rpm, so that again, as described in Example 1, an outer diameter of 0.35 mm resulted, albeit with the sheath having a larger thickness of layer.

### Example 6

### Coating a PET Multifilament Yarn from Solution with PVA/PVP in a Ratio of 30% by Weight/70% by Weight

60 g of Mowiol 44-88 polyvinyl alcohol (PVA) and 140 g of Luvitec K85 polyvinylpyrrolidone (PVP) were dissolved in 1000 ml of water at 80° C. while stirring. The solution was then cooled down to 50° C. A multifilament yarn of polyethylene terephthalate (PET) having a USP 2-0 thread size was pulled through this solution at a speed of 2 m/min. The arrangement of the change of direction rollers within and outwith the coating bath were chosen such that the coated thread departed from the bath surface at a right angle. After passing through a stripper system, the thread was dried by passing it through a heating duct 2 m in length and temperature controlled to 130° C. The coating applied was on average 15 μm in thickness.

### Example 7

### Terminal Dissolutive Removal of a Monofilament Sheath to Reduce the Ratio of Needle Diameter to Thread Diameter

The end of the core-sheath monofilament (USP 2-0) described in Example 5 was dipped into a hot water bath at 70° C. to an extent corresponding to the length of a drilled needle hole. After just a few minutes, the sheath of the monofilament had dissolved along the immersed length. The region tapered in this way was adaptable to a needle corresponding to the smaller diameter USP 3-0.

### Example 8

### Terminal Ablative Removal of a Monofilament Sheath for Educe the Ratio of Needle Diameter to Thread Diameter

To remove the sheath of the core-sheath monofilament (USP 2-0) described in Example 5 ablatively along a length corresponding to the depth of a drilled needle hole, a device as also used for removing insulation from electric cables was used. In a specific embodiment, the device was electrically heated to 180° C. This caused the monofilament sheath to melt, making it easier to remove. The tapered region was adaptable to a needle corresponding to the smaller diameter USP 3-0.

### Example 9

### Crosslinking a Monofilament Sheath by Exposure to β-Rays to Form a Hydrogel

The core-sheath monofilament described in Example 5 and the pseudomonofilament described in Example 6 were fed to an electron beam curing range (ESH 150 from Dürr) at a transportation speed of 4.5 m/min. Exposure to electron beam irradiation took place at an acceleration voltage of 180 kV. The dose rate was set at about 25 kGy by regulating the beam current. In a further experiment, the core-sheath monofilament of Example 5 and the pseudomonofilament of Example 6 were incipiently moistened before entry into the electron beam curing range. After crosslinking, pieces 50 cm in length were stored in distilled water at 70° C. for 3 hours to dissolve out uncrosslinked constituents. In all cases, distinct swelling of the sheath was observed. Subsequently, the thread pieces were dried in high vacuum at 80° C. for 24 hours and then weighed. The materials not incipiently moistened were found to have a 4% lower mass on average than the materials incipiently moistened before crosslinking, showing that crosslinking in the moist state proceeds at even greater efficiency.

1. A surgical thread that avoids puncture channel bleeding comprising a polymeric core and a polymeric, sheath surrounding the polymeric core, wherein the polymeric sheath is swellable in bodily fluids.

2. The surgical thread according to claim 1, wherein the polymeric core and the polymeric sheath touch along a common interface Without the core and the polymeric sheath being attached to each other by a covalent bond.

3. The surgical thread according to claim 1, wherein the polymeric core and the polymeric sheath are adhered together along a common interface.

4. The surgical thread according to claim 1, shaped as an extrusion thread, coextrusion thread, bicomponent thread or sheath extrusion thread.

5. The surgical thread according to claim 1, wherein the polymeric sheath has an absorbency for bodily fluids which corresponds to 3 to 80 times its own dry weight.

6. The surgical thread according to claim 1, wherein the polymeric sheath comprises additives swellable in bodily fluids.

7. The surgical thread according to claim 1, wherein the polymeric sheath includes additives swellable in bodily fluids in a proportion between 2% and 20% by weight based on the overall weight of the polymeric sheath.

8. The surgical thread according to claim 1, wherein the polymeric sheath includes a hydrophilic, water-insoluble and at least partly elastic polymer.

9. The surgical thread according to claim 8, wherein the polymer is selected from the group consisting of polyurethanes, polyester-ethers, mixtures thereof and copolymers thereof.

10. The surgical thread according to claim 1, wherein the polymeric sheath includes a polymer matrix swellable in bodily fluids.

11. The surgical thread according to claim 10, wherein the polymer matrix includes a hydrophilic, water-insoluble and at least partly elastic polymer and additives swellable in bodily fluids.

12. The surgical thread according to claim 1, wherein the polymeric sheath includes a polymer blend comprising a hydrophilic, water-insoluble and at least partly elastic polymer, and hydrophilic and water-soluble polymer.

13. The surgical thread according to claim 12, wherein the hydrophilic and preferably water-soluble polymer is selected from the group consisting of polyethylene glycol, polypropylene oxide, polytetramethylene oxide, polyvinylpyrrolidone, polyvinyl alcohol, mixtures thereof and copolymers thereof.

14. The surgical thread according to claim 1, wherein the polymeric sheath is a hydrogel or is convertible into a hydrogel on contact with bodily fluids.

15. The surgical thread according to claim 1, wherein the polymeric sheath includes a chemically and/or physically crosslinkable polymer.

16. The surgical thread according to claim 1, wherein the polymeric sheath includes a chemically and/or physically crosslinked, and water-insoluble polymer.

17. The surgical thread according to claim 15, wherein the polymer is selected from the group consisting of polyvinyl alcohol, polyvinylpyrrolidone, mixtures thereof and copolymers thereof.

18. The surgical thread according to claim 1, wherein the polymeric core is not swellable in bodily fluids.

19. The surgical thread according to claim 1, wherein the polymeric core is formed of a polymer from the group consisting of polyolefins, polyesters, polyamides, mixtures thereof and copolymers thereof.

20. The surgical thread according to claim 1, wherein the polymeric sheath has a fraction between 15% and 60% by volume based on the overall volume of the thread.

21. The surgical thread according to claim 1, wherein the polymeric sheath has a unitary thickness of layer.

22. The surgical thread according to claim 1, wherein the polymeric sheath has a radius fraction between 8% and 37% based on the overall radius of a cross-sectionally circular thread.

23. The surgical thread according to claim 1, which is a monofilament, multifilament, or pseudomonofilament.

24. The surgical thread according to claim 1, having a tapered diameter in a region of one end or both ends.

25. A surgical kit comprising a surgical thread according to claim 1 and at least one surgical needle.

26. A method for producing a surgical thread, according to any one of claim 1, wherein a polymeric thread core component and a polymeric sheath component swellable in bodily fluids are coextruded to form threads having a polymeric core and a polymeric sheath swellable in bodily fluids which surrounds the core.

27. A method for producing a surgical thread, according to claim 1, wherein a thread-shaped polymeric thread core component is coated by sheath extrusion with a polymeric sheath component swellable in bodily fluids to form threads having a polymeric core and a polymeric sheath swellable in bodily fluids which surrounds the core.

28. A method for producing a surgical thread, according to claim 1, wherein a thread-shaped, polymeric thread core component is dipped into an aqueous solution of a crosslinkable, water-soluble and polymeric sheath component, pulled through an aqueous solution of a crosslinkable, water-soluble and polymeric sheath component or sprayed with a solution of a crosslinkable, water-soluble and polymeric sheath component, and the sheath component is crosslinked to form threads having a polymeric core and a polymeric sheath swellable in bodily fluids which surrounds the core.

29. The method according to claim 26, wherein a hydrophilic water-insoluble and at least partly elastic polymer is used for the sheath component.

30. The method according to claim 26, wherein swellable additives are incorporated into a hydrophilic, water-insoluble and at least partly elastic polymer to provide the sheath component.

31. The method according to claim 30, wherein the swellable additives are incorporated in the polymer as a masterbatch or compound.

32. The method according to claim 26, wherein the sheath component is subjected to chemical and/or physical crosslinking.

33. The method according to claim 26, wherein the crosslinking is carried out with a crosslinking agent selected from the group consisting of carbodiimides, divinyl sulfone, N-methylenebisacrylamide, epichlorohydrin, bi- or oligofunctional aldehydes, polyfunctional polyaldehydes, di- or oligofunctional carboxylic acids, di- or oligofunctional esters, polyfunctional polycarboxylic acids, polyfunctional polyesters and borax.

34. The method according to claim 26, wherein crosslinking is carried out free-radically under action of UV light and in the presence of free-radical initiators.

35. The method according to claim 26, wherein the crosslinking is effected under action of ionizing radiation.

36. The method according to claim 28, wherein crosslinking of the sheath component is carried out in the aqueous solution.

37. (canceled)
